(19) United States
(12) Patent Application Publication
(10) Pub. No.: US 2014/0294991 A1
(43) Pub. Date: Oct. 2, 2014

(54) MEDICAMENT FOR TREATING ANTERIOR EYE DISEASE COMPRISING REBAMIPIDE AND A TEAR-RETAINING AGENT

(71) Applicant: OTSUKA PHARMACEUTICAL CO., LTD., Chiyoda-ku, Tokyo (JP)

(72) Inventors: Yasuhiro Takeji, Osaka-shi (JP); Hideo Nakashima, Osaka-shi (JP); Hiroki Urashima, Osaka-shi (JP); Hisashi Shinohara, Osaka-shi (JP); Yuki Hirata, Osaka-shi (JP)

(21) Appl. No.: 14/355,375

(22) PCT Filed: Oct. 31, 2012
(86) PCT No.: PCT/JP2012/078769

§ 371 Date: Apr. 30, 2014

(30) Foreign Application Priority Data
Nov. 1, 2011 (JP) 2011-240177

(51) Int. Cl. A61K 31/47; A61K 31/728; A61K 31/737; A61K 33/30

(52) U.S. Cl. CPC A61K 31/47; A61K 33/30; A61K 31/728; A61K 31/737
USPC 424/641; 514/54

(57) ABSTRACT

The present invention provides a combination of rebamipide and a tear-retaining agent as a medicament for the treatment of anterior eye diseases.

### TECHNICAL FIELD

The present invention relates to a medicament for the treatment of an anterior eye disease comprising rebamipide and a tear-retaining agent or an artificial tear.

### BACKGROUND ART

The surface of the cornea or conjunctiva of patients suffering from anterior eye diseases including corneal diseases and conjunctival diseases such as dry eye is damaged due to various reasons. In particular, various factors in dry eye such as decreased tear volume or tear evaporation can cause eye dryness (so-called “dry eye”) and damage to the eye. Thus, dry eye has a risk of causing corneal ulcer or visual loss if left untreated, and various therapies are clinically used. Methods for curing the lesions caused by dry eye include the following three types; medical therapies, punctal plugs, and surgeries. A drug having a tear-retention capacity such as sodium hyaluronate, a drug which increases the conjunctival mucin level such as diquafosol tetrasodium, and an artificial tear mimicking the composition of natural tears are used for the medical therapy of dry eye.

Recently, rebamipide (2-(4-chlorobenzoylamino)-3-[2(1H)-quinolin-4-yl]propionic acid) which promotes mucin secretion from corneal epithelial cells and conjunctival goblet cells and proliferation of goblet cells was developed as a novel drug based on a novel mode of action. Rebamipide cures damages on the surface of the cornea and conjunctiva and alleviates subjective symptoms in dry eye by promoting mucin secretion from corneal epithelial cells and conjunctival goblet cells to stabilize the tear fluid and improving corneal epithelial damage (Patent Reference 1).

Sodium hyaluronate which assists tear retention and improves damages on the cornea is commercially available as Hyalein® 0.1% and 0.3% (Santen Pharmaceutical Co., Ltd.).

Many artificial tear productions which mimic the composition of natural tears for improving dryness of the eye, foreign body sensation and the like are commercially available.

There is no report that anterior eye diseases such as dry eye are/were treated with a combination of rebamipide or a salt thereof and an agent having a tear retention capacity or an artificial tear.

### PRIOR ART DOCUMENTS

[Patent Reference 1] WO 1997/013515

### SUMMARY OF INVENTION

Evaluating the utility of such combination of rebamipide having the novel action or a salt thereof and a tear-retaining agent or an artificial tear as a medicament for the treatment of anterior eye diseases is an absolutely attractive issue.

The present inventors have intensively studied the possibility of developing a combination of rebamipide and a tear-retaining agent or an artificial tear as a medicament for the treatment of anterior eye diseases such as dry eye, and have found that the tear retention capacity at the ocular surface and the improvement of corneal epithelial damage can be enhanced by such combination. Based upon the new findings, the present invention has been completed. It was demonstrated that such combination of rebamipide and a tear-retaining agent markedly enhance the tear retention capacity at the ocular surface and the improvement of corneal epithelial damage. The detail of the experimental methods and the result will be described in the section of the pharmacological experiments below. Furthermore, the present medicament for the treatment of an anterior eye disease can also be conveniently used for the prevention of dry eye and the treatment of diseases besides dry eye in which the cornea or conjunctiva is damaged.

The present invention encompasses the following aspects.

[1] A medicament for the treatment of an anterior eye disease comprising a combination of rebamipide or a salt thereof, and a tear-retaining agent or an artificial tear.

[2] A medicament for the treatment of an anterior eye disease comprising a combination of rebamipide or a salt thereof, and a tear-retaining agent or an artificial tear, wherein the effect of each ingredient is mutually supplemented and/or enhanced.

[3] The medicament for the treatment of an anterior eye disease described in [1] or [2] comprising a combination of rebamipide or a salt thereof, and a tear-retaining agent.

[4] A medicament for the treatment of an anterior eye disease comprising rebamipide or a salt thereof in a combination with a tear-retaining agent.

[5] The medicament for the treatment of an anterior eye disease described in [3] or [4] wherein the tear-retaining agent is hyaluronic acid or a salt thereof, or chondroitin sulfuric acid or a salt thereof.

[6] The medicament for the treatment of an anterior eye disease described in any one of [1] to [5] wherein the anterior eye disease is a corneal disease or a conjunctival disease.

[7] The medicament for the treatment of an anterior eye disease described in any one of [1] to [5] wherein the anterior eye disease is dry eye.

[8] An ophthalmic solution comprising rebamipide or a salt thereof, and hyaluronate.

[9] The ophthalmic solution described in [8] further comprising a zinc compound.

[10] The ophthalmic solution described in [9] wherein the zinc compound is zinc chloride and/or zinc sulfate.

[11] The ophthalmic solution described in any one of [8] to [10] further comprising a solubilizer, an amino acid, and a buffer.

[12] The ophthalmic solution described in any one of [8] to [11] further comprising an isotonic agent.

[13] The ophthalmic solution described in any one of [8] to [12] wherein the pH is 7 to 9.

[14] The ophthalmic solution described in [12] or [13] wherein the concentration of the zinc compound is 0.000001% (w/v) to 0.0001% (w/v) in terms of the concentration of zinc.

[15] The ophthalmic solution described in any one of [8] to [14] wherein the concentrations of rebamipide and hyaluronate are 1 (w/v) to 3% (w/v) and 0.1 (w/v) to 0.3% (w/v), respectively.

[16] A method for the treatment of an anterior eye disease comprising administrating a combination of rebamipide or a salt thereof, and a tear-retaining agent or an artificial tear to a patient in need of such treatment.

[17] A combination of rebamipide or a salt thereof, and a tear-retaining agent or an artificial tear for the treatment of an anterior eye disease.

### BRIEF DESCRIPTION OF DRAWINGS

FIG. 1 shows a graph of the retained tear volume for each solution of the test compounds in Pharmacological Experiment 1.

FIG. 2 shows a graph of the corneal epithelial damage (scores) for each solution of the test compounds in Pharmacological Experiment 1.

FIG. 3 shows a graph of the retained tear volume for each solution of the test compounds in Pharmacological Experiment 2.

FIG. 4 shows a graph of the corneal epithelial damage (scores) for each solution of the test compounds in Pharmacological Experiment 2.

FIG. 5 shows a graph of the retained tear volume for each solution of the test compounds in Pharmacological Experiment 3.

FIG. 6 shows a graph of the corneal epithelial damage (scores) for each solution of the test compounds in Pharmacological Experiment 3.

### DESCRIPTION OF EMBODIMENTS

The present invention provides a medicament for the treatment of anterior eye disease(s) such as dry eye comprising a combination of rebamipide or a salt thereof, and a tear-retaining agent or an artificial tear, wherein the effect of each ingredient can be mutually supplemented and/or enhanced.

For the treatment of an anterior eye disease, rebamipide or a salt thereof, and a tear-retaining agent or an artificial tear may be combined and administrated as a single formulation (i.e., a drug combination), or may be separately formulated and separately administrated (i.e., a combined administration).

As the salt of rebamipide, a physiologically or pharmaceutically acceptable salt of rebamipide can be used. Examples of the salt include a salt formed with a typical base such as sodium hydroxide, potassium hydroxide, trometamol (tris[hydroxymethyl]aminomethane), monoethanol-amine, diethanolamine, triethanolamine, diisopropanolamine, meglumine or the like.

The tear-retaining agent may be in a form of a salt. Examples of the salt include a salt of an inorganic acid with a base such as sodium and potassium, in particular, a sodium salt is preferable.

The present invention is characterized by using the combination of rebamipide or a salt thereof, and a tear-retaining agent or an artificial tear for the treatment of an anterior eye disease. The tear-retaining agent used herein is not limited to a specific one as long as it has a tear-retention capacity or a tear-supplementation capacity and is useful for the treatment of an anterior eye disease. Examples of the tear-retaining agent include sodium hyaluronate, sodium chondroitin sulfate and the like, and in particular, sodium hyaluronate which is already commercially available is preferable. These tear-retaining agents, as a matter of course, may or may not be in a form of a salt or ester.

The artificial tears comprise ingredients which are similar to those of natural tears. Various artificial tears which can be utilized for the present invention are commercially available.

For carrying out the present invention, rebamipide or a salt thereof, and a tear-retaining agent or an artificial tear may be combined to a single formulation, or may be formulated to separate formulations. These formulations can be prepared by using a conventional technique in the art without requiring any special technique. The preferred modes of administration include topical administration, and the preferred dosage forms include an eye drop, eye ointment and the like.

Rebamipide or a salt thereof, and a tear-retaining agent or an artificial tear can be separately formulated according to a well-known technique. For example, formulations of rebamipide disclosed in WO 2009/154304, WO 2008/050896 and WO 2006/052018, or commercially available rebamipide products can be used for the present invention. The formulation of a tear-retaining agent or an artificial tear can be prepared by reference to the above-mentioned patent publications. Commercially available Hyalein® (Santen Pharmaceutical Co., Ltd.), Chondron® (Kaken Pharmaceutical Co., Ltd.), and Tearbalance® (Senju Pharmaceutical Co., Ltd.) which have been already on the market as a medicament for the treatment of an anterior eye disease may be used as the formulation of a tear-retaining agent or an artificial tear.

The formulations comprising rebamipide or a salt thereof, and a tear-retaining agent or an artificial tear can be prepared according to well-known techniques. When the formulation is in a form of an eye drop, a zinc compound such as zinc chloride and zinc sulfate; a solubilizer such as polyvinylpyrrolidone; an amino acid such as Meglumine; an isotonic agent such as sodium chloride, concentrated glycerin and the like; a buffer such as sodium phosphate, sodium acetate, boric acid and the like; a surfactant such as polyoxyethylene sorbitan monooleate, polyoxyl 40 stearate, polyoxyethylene hydrogenated caster oil and the like; a stabilizer such as sodium citrate, sodium edetate and the like; a suspending agent such as polyvinyl alcohol; a pH adjusting agent such as hydrochloric acid, sodium hydroxide and the like; a preservative such as benzalkonium chloride, paraben, zinc and the like may be used if needed. The pH of the formulation should be in an ophthalmologically acceptable range, and preferably in the range of 4 to 9, more preferably 7 to 9. When the present invention is prepared in an ophthalmic solution comprising a zinc compound, the concentration of the zinc compound is 0.000001% (w/v) to 0.0001% (w/v), preferably 0.000003% (w/v) to 0.0001% (w/v) in terms of the concentration of zinc. Non-limiting examples of the formulation will be described in the section of working examples below.

The dosage of rebamipide or a salt thereof, and a tear-retaining agents or an artificial tear will be determined according to the symptoms, ages of the patients, the dosage form, the route of administration and the like. For example, rebamipide is topically administrated in a daily dosage of 0.2 to 8 mg per one eye in a single shot or divided shots of several times, preferably 4 to 6 times a day. The dosage of the tear-retaining agent can vary depending on the type of the agent, and should be determined based on the standard dosage which is clinically used, which can be optionally adjusted depending on the objective symptoms and the like. The daily dosage is from 20 to 2000 μg per one eye in a single shot or divided shots of several times. For example, the daily dosage of 100 to 1500 μg for sodium hyaluronate is generally employed, however, the dosage may be optionally adjusted depending on the objective symptoms and the like. The dosage of other tear-retaining agents can be determined similarly. These dosages are employed when rebamipide or a salt thereof, and a tear-retaining agent or an artificial tear are administrated as a combined administration. When rebamipide and a tear-retaining agent or an artificial tear are combined to a single formulation, the composition rate of each ingredient in the formulation is adjusted so that the daily amount for topical administration is equal to or less than the amount of each ingredient described above, and the formulation is topically administrated in a single shot or divided shots of several times daily.

When the present invention is prepared in an ophthalmic solution comprising rebamipide and hyaluronate, the concentrations of rebamipide and hyaluronate are 1 (w/v) to 3% (w/v) and 0.05 (w/v) to 0.4% (w/v), preferably 1.5 (w/v) to 2.5% (w/v) and 0.1 (w/v) to 0.3% (w/v), respectively.

### EXAMPLES

Hereinafter, the present invention is illustrated by the following examples of the formulations and pharmacological experiments, but should not be construed to be limited thereto.

A typical example of an eye drop in the present invention comprising rebamipide or a salt thereof, and a tear-retaining agent is shown below.

### Formulation Example 1

To a solution of sodium hyaluronate (0.1 g), polyvinyl alcohol (1 g), sodium chloride (0.8 g) and sodium citrate (0.2 g) in purified water (q.s.) was added rebamipide (2 g), and then purified water was added thereto to prepare 100 mL of a suspension.

### Formulation Example 2

Rebamipide (20 g), polyvinylpyrrolidone (30 g), meglumine (42 g), boric acid (10 g), zinc chloride (0.00104 g), sodium hyaluronate (1 g), and glycerin (12 g) were dissolved in purified water (q.s.), and then purified water was further added thereto to prepare 1000 mL of a solution.

### Formulation Example 3

Rebamipide (20 g), polyvinylpyrrolidone (30 g), meglumine (42 g), boric acid (10 g), zinc chloride (0.001456 g), sodium hyaluronate (1 g), and glycerin (12 g) were dissolved in purified water (q.s.), and then purified water was further added thereto to prepare 1000 mL of a solution.

### Formulation Example 4

Rebamipide (20 g), polyvinylpyrrolidone (30 g), meglumine (42 g), boric acid (10 g), zinc chloride (0.00208 g), sodium hyaluronate (1 g), and glycerin (12 g) were dissolved in purified water (q.s.), and then purified water was further added thereto to prepare 1000 mL of a solution.

### Formulation Example 5

Rebamipide (20 g), polyvinylpyrrolidone (30 g), meglumine (42 g), boric acid (10 g), zinc chloride (0.00208 g), and sodium hyaluronate (1 g) were dissolved in purified water (q.s.), and then purified water was further added thereto to prepare 1000 mL of a solution.

The antimicrobial effectiveness tests according to the Japanese. Pharmacopoeia and the US Pharmacopoeia were carried out using Formulation Examples 1, 3 and 4, and the results were all acceptable.

### Pharmacological Experiment 1

To evaluate the effect of a combination of rebamipide and a tear-retaining agent or an artificial tear, rebamipide and sodium hyaluronate were administrated in combination to a mouse model for dry eye, and the tear volume at the ocular surface was measured and the corneal epithelial damage was evaluated.

### (Solution of the Test Compound)

Rebamipide was administrated as a 2% rebamipide eye drop (an eye drop comprising 2% rebamipide). Sodium hyaluronate was administrated as Hyalein Mini® ophthalmic solution 0.1% (an eye drop comprising a 0.1 sodium hyaluronate solution).

### (Control Model Group)

One control model group was used as non dry eye group.

### (Dry Eye Model Group)

Dry eye model groups were used as four groups: i.e., “non-treatment” group, “rebamipide-treated” group, “sodium hyaluronate-treated” group and “rebamipide+sodium hyaluronate-treated” group.

### (Preparation of a Mouse Model for Dry Eye)

The mouse model for dry eye was prepared by daily subcutaneous administration of a solution of scopolamine (a parasympatholytic) in saline to C57BL mice in a dose of 0.5 mg/0.1 mL/individual for four times per day.

### Mode of Administration

Daily, 2 μl of each drug solution for one eye was topically administrated to the mice of the “2% rebamipide-treated” group (4 times/day), “0.1 sodium hyaluronate-treated” group (6 times/day), and “2% rebamipide (4 times/day)+0.1% sodium hyaluronate (6 times/day)-treated” group. For the combination administration, 0.1% sodium hyaluronate was administrated at least 5 minutes after the administration of 2% rebamipide.

### (Measurement of Tear Volumes)

The tear volumes were measured as an index of a tear-retention capacity by the phenol red thread test. At the sixth day from the beginning of the administration of the test compound solution, a cotton thread was placed in the temporal conjunctiva of the mouse, and the tear volume 10 minutes after the topical administration was measured for 30 seconds.

The tear volume (mm) was defined as the length of the part of the cotton thread where color changed by the tear.

### (Evaluation of Corneal Epithelial Damage)

Corneal epithelial damage was evaluated by applying sodium fluorescein under a blue filter. At the sixth day from the beginning of the administration of the test compound solution, 1 μl of 1% sodium fluorescein solution was topically applied to the eye, and then the eyes were washed with saline. The degree of corneal staining was graded as 0 to 9.

### (Result)

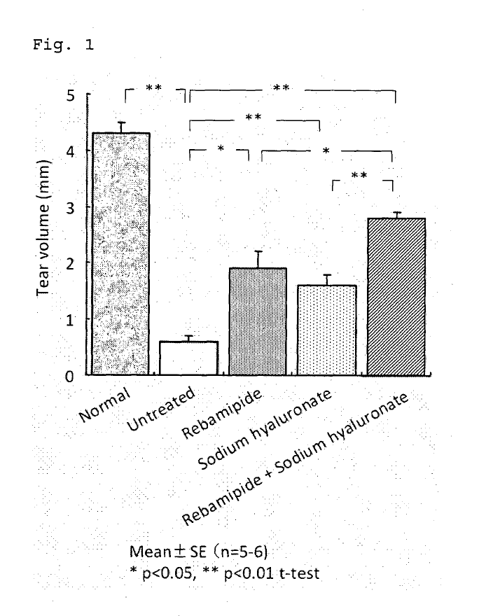

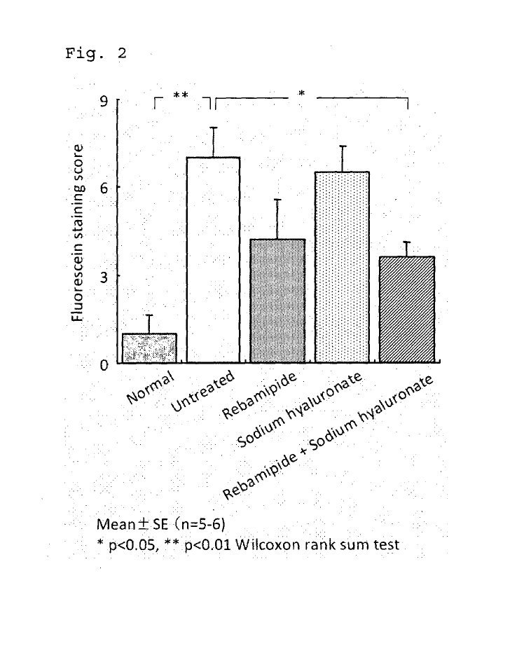

The results are shown in FIGS. 1 and 2.

### (Discussion)

As shown in FIG. 1, the tear volume of the “rebamipide+sodium hyaluronate-treated” group was significantly higher than those of the “rebamipide-treated” group and the “sodium hyaluronate-treated” group. Furthermore, as shown in FIG. 2, the corneal epithelial damage in the “rebamipide+sodium hyaluronate-treated” group was significantly improved when compared to the “non-treated” group. These results demonstrate that the combination of rebamipide and sodium hyaluronate provides superior tear retention and marked improvement of the corneal epithelial damages.

### Pharmacological Experiment 2

In the same manner as Pharmacological Experiment 1, a drug combination of rebamipide and sodium hyaluronate was administrated, and the tear volume at the ocular surface was measured and the corneal epithelial damage was evaluated.

### (Solution of the Test Compound)

Rebamipide was administrated as a 2% rebamipide eye drop (an eye drop comprising 2% rebamipide). Sodium hyaluronate was administrated as Hyalein Mini® ophthalmic solution 0.1% (an eye drop comprising a 0.1% sodium hyaluronate solution). As for the administration of a drug combination, the drug combination prepared in Formulation Example 4 was used.

### (Control Model Group)

One control model group was used as non dry eye group.

### (Dry Eye Model Group)

Dry eye model groups were used as four groups: i.e., “non-treatment” group, “rebamipide-treated” group, “sodium hyaluronate-treated” group and “rebamipide+sodium hyaluronate-treated” group (using the drug combination prepared in Formulation Example 4).

### (Preparation of a Mouse Model for Dry Eye)

The mouse model for dry eye was prepared by daily subcutaneous administration of a solution of scopolamine (a parasympatholytic) in saline to C57BL mice in a dose of 0.5 mg/0.1 mL/individual for four times per day.

### (Mode of Administration)

Daily, 2 μl of each drug solution for one eye was topically administrated to the mice of the “2% rebamipide-treated” group (4 times/day), “0.1% sodium hyaluronate-treated”group (6 times/day), and “rebamipide+sodium hyaluronate-treated” group (4 times/day).

### (Measurement of Tear Volumes)

The tear volumes were measured as an index of a tear-retention capacity by the phenol red thread test. At the sixth day from the beginning of the administration of the test compound solution, a cotton thread was placed in the temporal conjunctiva of the mouse, and the tear volume 10 minutes after the topical administration was measured for 30 seconds.

The tear volume (mm) was defined as the length of the part of the cotton thread where color changed by the tear.

### (Evaluation of Corneal Epithelial Damage)

Corneal epithelial damage was evaluated by applying sodium fluorescein under a blue filter. At the sixth day from the beginning of the administration of the test compound solution, 1 μl of 1% sodium fluorescein solution was topically applied to the eye, and then the eyes were washed with saline. The degree of corneal staining was graded as 0 to 9.

### (Result)

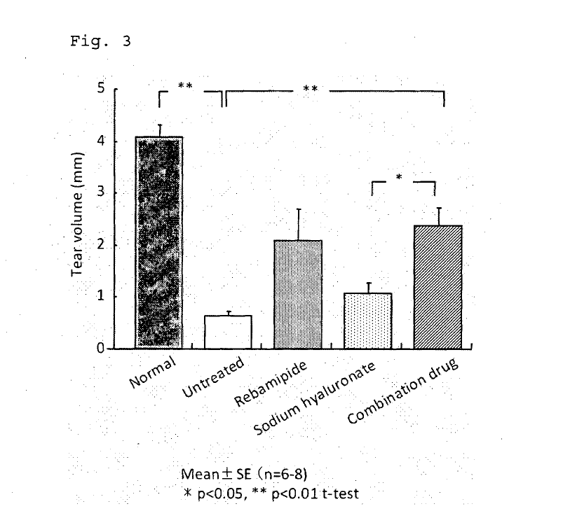

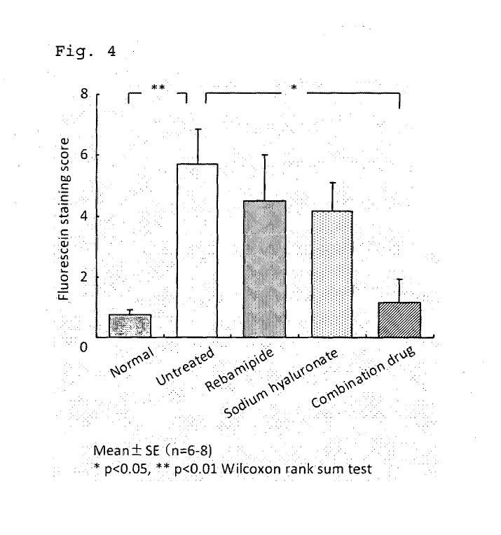

The results are shown in FIGS. 3 and 4.

### (Discussion)

As shown in FIG. 3, the tear volume of the “rebamipide+sodium hyaluronate-treated” group was significantly higher than that of the “non-treated” group. Furthermore, as shown in FIG. 4, the corneal epithelial damage in the “rebamipide+sodium hyaluronate-treated” group was significantly improved when compared to the “non-treated” group. These results demonstrate that the drug combination of rebamipide and sodium hyaluronate provides superior tear retention and marked improvement of the corneal epithelial damages.

### Pharmacological Experiment 3

To evaluate the utility of a combination of rebamipide and a tear-retaining agent or an artificial tear, rebamipide and sodium hyaluronate were administrated in combination to a tear deficient rat model, and the tear volume was measured and the corneal epithelial damage was evaluated. The tests were carried out in single administration and repeated administration.

### (Solution of the Test Compound)

Rebamipide was administrated as a 2% rebamipide eye drop (an eye drop comprising 2% rebamipide). Sodium hyaluronate was administrated as Hyalein Mini® ophthalmia solution 0.1% (an eye drop comprising a 0.1% sodium hyaluronate solution).

### (Control Model Group)

One control model group was used as non-decreased tear volume group.

### (Tear Deficient Rat Model)

Tear deficient rat model groups were used as four groups: i.e., “non-treatment” group, “rebamipide-treated” group, “sodium hyaluronate-treated” group and “rebamipide+sodium hyaluronate-treated” group.

### (Preparation of a Tear Deficient Rat Model)

Capsaicin (50 mg/kg) was administered to a 4-day-old Wister/ST rat, and 4 weeks later the rat was used as a tear deficient rat model.

### (Mode of Administration)

For the single administration test, 5 μl of each drug solution for one eye was topically administrated to the rats of the “2% rebamipide-treated” group, “0.1% sodium hyaluronate-treated” group, and “2% rebamipide+0.1% sodium hyaluronate-treated” group. For the combination administration, 0.1% sodium hyaluronate was administrated 5 minutes after the administration of 2% rebamipide.

For the repeated administration test, 5 μl of each drug solution for one eye was topically administrated to the rats of the “2% rebamipide-treated” group (4 times/day), “0.1% sodium hyaluronate-treated” group (6 times/day), and “2% rebamipide (4 times/day)+0.1% sodium hyaluronate (6 times/day)-treated” group. For the combination administration, 0.1% sodium hyaluronate was administrated at least 5 minutes after the administration of 2% rebamipide.

### (Measurement of Tear Volumes)

The tear volumes were measured as an index of a tear-retention capacity by Schirmer's test. A schirmer paper (1.5 mm) was placed in the inferior conjunctiva of the rat, and the tear volume was measured for one minute.

### (Evaluation of Corneal Epithelial Damage)

Corneal epithelial damage was evaluated by sodium fluorescein under a blue filter. At the tenth day from the beginning of the administration of the test compound solution, 1 μl of 1% sodium fluorescein solution was topically applied to the eye, and then the eyes were washed with saline. The degree of corneal staining was graded as 0 to 9.

### (Result)

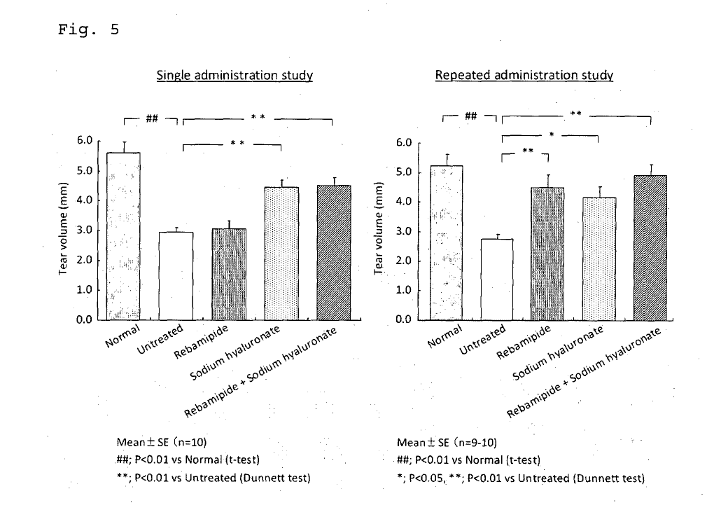

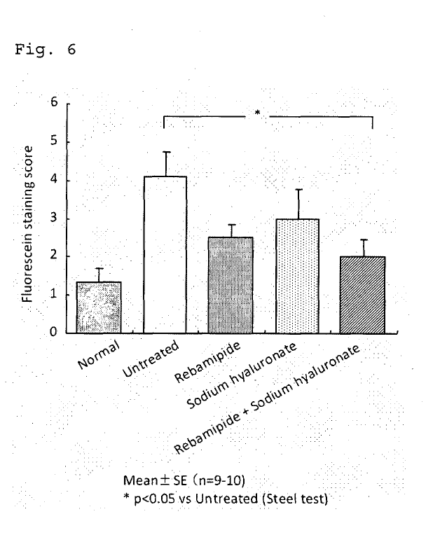

The results are shown in FIGS. 5 and 6.

### (Discussion)

As shown in FIG. 5, for the single administration test, the tear volumes of the “rebamipide+sodium hyaluronate-treated” group and the “sodium hyaluronate-treated” group were significantly higher than that of the “non-treated” group. For the repeated administration test, the tear volumes of the “rebamipide+sodium hyaluronate-treated” group, the “sodium hyaluronate-treated” group and the “rebamipide-treated” group” were significantly higher than that of the “non-treated” group. Furthermore, as shown in FIG. 6, the corneal epithelial damage in the “rebamipide+sodium hyaluronate-treated” group was significantly improved when compared to the “non-treated” group. These results demonstrate that the combination of rebamipide and sodium hyaluronate provides rapid-acting tear retention and marked improvement of the corneal epithelial damages.

### INDUSTRIAL APPLICABILITY

A combination of rebamipide and a tear-retaining agent such as sodium hyaluronate or an artificial tear provided superior tear retention and marked improvement of the corneal epithelial damages. Thus, the present combination of rebamipide and a tear-retaining agent such as sodium hyaluronate or an artificial tear is useful for the treatment of an anterior eye disease such as dry eye.

1. A medicament for the treatment of an anterior eye disease comprising a combination of rebamipide or a salt thereof, and a tear-retaining agent or an artificial tear.

2. A medicament for the treatment of an anterior eye disease comprising a combination of rebamipide or a salt thereof, and a tear-retaining agent or an artificial tear, wherein the effect of each ingredient is mutually supplemented and/or enhanced.

3. The medicament for the treatment of an anterior eye disease of claim 1 or 2 comprising a combination of rebamipide or a salt thereof, and a tear-retaining agent.

4. A medicament for the treatment of an anterior eye disease comprising rebamipide or a salt thereof in a combination with a tear-retaining agent.

5. The medicament for the treatment of an anterior eye disease of claim 3 or 4 wherein the tear-retaining agent is hyaluronic acid or a salt thereof, or chondroitin sulfuric acid or a salt thereof.

6. The medicament for the treatment of an anterior eye disease of any one of claims 1-5 wherein the anterior eye disease is a corneal disease or a conjunctival disease.

7. The medicament for the treatment of an anterior eye disease of any one of claims 1-5 wherein the anterior eye disease is dry eye.

8. An ophthalmic solution comprising rebamipide or a salt thereof, and hyaluronate.

9. The ophthalmic solution of claim 8 further comprising a zinc compound.

10. The ophthalmic solution of claim 9 wherein the zinc compound is zinc chloride and/or zinc sulfate.

11. The ophthalmic solution of any one of claims 8 to 10 further comprising a solubilizer, an amino acid, and a buffer.

12. The ophthalmic solution of any one of claims 8 to 11 further comprising an isotonic agent.

13. The ophthalmic solution of any one of claims 8 to 12 wherein the pH is 7 to 9.

14. The ophthalmic solution of claim 12 or 13 wherein the concentration of the zinc compound is 0.000001% (w/v) to 0.0001% (w/v) in terms of the concentration of zinc.

15. The ophthalmic solution of any one of claims 8 to 14 wherein the concentrations of rebamipide and hyaluronate are 1 (w/v) to 3% (w/v) and 0.1 (w/v) to 0.3% (w/v), respectively.
